SEQUENCE LISTING (28 sequences)

SEQ ID NO: 1 - protein, 15 aa, Artificial Sequence
  Completely synthesized peptide construct
EKAGVVSTGLIQNGD

SEQ ID NO: 2 - protein, 15 aa, Artificial Sequence
  peptide construct
  MISC_FEATURE (1)..(1): acetyl, ClAc, BrAc
  MISC_FEATURE (1)..(1): X is A or N and when X is A, then A is either cyclohexylalanine or D-alanine
  MISC_FEATURE (15)..(15): highly variable fragment wherein isoleucine at 143 and leucine at 159 in contact with CD4 phenylalanine as the 43 residue
  MOD_RES (15)..(15): AMIDATION
XGQEEKAGVVSTGLI

SEQ ID NO: 3 - protein, 11 aa, Artificial Sequence
  Completely synthesized peptide construct
  MOD_RES (1)..(1): ACETYLATION, Aad
  MISC_FEATURE (1)..(1): acetyl, ClAc, BrAc
  MISC_FEATURE (1)..(1): X is A or E and when X is A, then A is either cyclohexylalanine or D-alanine
  MOD_RES (11)..(11): AMIDATION
XETVGVSQLEV

SEQ ID NO: 4 - protein, 15 aa, Artificial Sequence
  Completely synthesized peptide construct
NGQEETVGVSSTQLI

SEQ ID NO: 5 - protein, 18 aa, Artificial Sequence
  Completely synthesized peptide construct
NGQEEKAGVVSTGLIGGG

SEQ ID NO: 6 - protein, 14 aa, Artificial Sequence
  Completely synthesized peptide construct
  MISC_FEATURE (1)..(1): cyclohexylalanine, D-alanine, acetyl, ClAc, BrAc
  MISC_FEATURE (1)..(1): Xaa is an amino acid V, L, I, G or A
  MISC_FEATURE (4)..(4): Xaa is an amino acid V, L, I, G or A
  MISC_FEATURE (6)..(6): Xaa is an amino acid V, L, I, G or A
XETXGXSQLEVGGG

SEQ ID NO: 7 - protein, 18 aa, Artificial Sequence
  Completely synthesized peptide construct
  MISC_FEATURE (1)..(1): acetyl, ClAc, BrAc
  MISC_FEATURE (1)..(1): X is A or D and when X is A, then A is either cyclohexylalanine or D-alanine
  MISC_FEATURE (6)..(6): Xaa is an amino acid V, L, I, G or A
  MOD_RES (18)..(18): AMIDATION
XGQEEXAGVVSTGLIGGG

SEQ ID NO: 8 - protein, 17 aa, Artificial Sequence
  Completely synthesized peptide construct
  MISC_FEATURE (1)..(1): acetyl, ClAc, BrAc
  MISC_FEATURE (1)..(1): X is A or G and when X is A, then A is either cyclohexylalanine or D-alanine
  MOD_RES (17)..(17): AMIDATION
XQEEKAGVVSTGLIGGG

SEQ ID NO: 9 - protein, 18 aa, Artificial Sequence
  Completely synthesized peptide construct
  MISC_FEATURE (1)..(1): acetyl, ClAc, BrAc
  MISC_FEATURE (1)..(1): X is A or D and when X is A, then A is either cyclohexylalanine or D-alanine
  MISC_FEATURE (6)..(6): Xaa is an amino acid V, L, I, G, A, K or F
  MISC_FEATURE (6)..(6): analogues such as substituted epsilon amino (methyl, alkyl) Lysine or hydroxy-Leucine or Leucine
  MOD_RES (18)..(18): AMIDATION
XGQEEXAGVVSTGLIGGG

SEQ ID NO: 10 - protein, 18 aa, Artificial Sequence
  Completely synthesized peptide construct
  MISC_FEATURE (1)..(1): acetyl, ClAc, BrAc
  MISC_FEATURE (1)..(1): X is A or D and when X is A, then A is either cyclohexylalanine or D-alanine
  MOD_RES (18)..(18): AMIDATION
XGQEEFAGVVSTGLIGGG

SEQ ID NO: 11 - protein, 10 aa, Artificial Sequence
  Completely synthesized peptide construct
  MISC_FEATURE (1)..(1): acetyl, ClAc, BrAc
  MISC_FEATURE (1)..(1): X is A or E and when X is A, then A is either cyclohexylalanine or D-alanine
  MOD_RES (10)..(10): AMIDATION
XTVGVSQLEV

SEQ ID NO: 12 - protein, 10 aa, Artificial Sequence
  Completely synthesized peptide construct
  MISC_FEATURE (1)..(1): acetyl, ClAc, BrAc
  MISC_FEATURE (1)..(1): X is A or E and when X is A, then A is either cyclohexylalanine or D-alanine
  MOD_RES (10)..(10): AMIDATION
XTVGVSQLEV

SEQ ID NO: 13 - protein, 9 aa, Artificial Sequence
  Completely synthesized peptide construct
  MISC_FEATURE (1)..(1): acetyl, ClAc, BrAc
  MISC_FEATURE (1)..(1): X is A or E and when X is A, then A is either cyclohexylalanine or D-alanine
  MOD_RES (9)..(9): AMIDATION
XTVGVSQLE

SEQ ID NO: 14 - protein, 8 aa, Artificial Sequence
  Completely synthesized peptide construct
  MISC_FEATURE (1)..(1): acetyl, ClAc, BrAc
  MISC_FEATURE (1)..(1): X is A or E and when X is A, then A is either cyclohexylalanine or D-alanine
  MOD_RES (8)..(8): AMIDATION
XTVGVSQL

SEQ ID NO: 15 - protein, 7 aa, Artificial Sequence
  Completely synthesized peptide construct
  MISC_FEATURE (1)..(1): acetyl, ClAc, BrAc
  MISC_FEATURE (1)..(1): X is A or E and when X is A, then A is either cyclohexylalanine or D-alanine
  MOD_RES (7)..(7): AMIDATION
XTVGVSQ

SEQ ID NO: 16 - protein, 6 aa, Artificial Sequence
  Completely synthesized peptide construct
  MISC_FEATURE (1)..(1): acetyl, ClAc, BrAc
  MISC_FEATURE (1)..(1): X is A or E and when X is A, then A is either cyclohexylalanine or D-alanine
  MOD_RES (6)..(6): AMIDATION
XTVGVS

SEQ ID NO: 17 - protein, 5 aa, Artificial Sequence
  Completely synthesized peptide construct
  MISC_FEATURE (1)..(1): acetyl, ClAc, BrAc
  MISC_FEATURE (1)..(1): X is A or E and when X is A, then A is either cyclohexylalanine or D-alanine
  MOD_RES (5)..(5): AMIDATION
XTVGV

SEQ ID NO: 18 - protein, 15 aa, Artificial Sequence
  Completely synthesized peptide construct
  MISC_FEATURE (1)..(1): acetyl, ClAc, BrAc
  MISC_FEATURE (1)..(1): X is A or D and when X is A, then A is either cyclohexylalanine or D-alanine
XGQEEKAGVVSTGLI

SEQ ID NO: 19 - protein, 14 aa, Artificial Sequence
  Completely synthesized peptide construct
  MISC_FEATURE (1)..(1): acetyl, ClAc, BrAc
  MISC_FEATURE (1)..(1): X is A or G and when X is A, then A is either cyclohexylalanine or D-alanine
XQEEKAGVVSTGLI

SEQ ID NO: 20 - protein, 19 aa, Artificial Sequence
  Completely synthesized peptide construct
  MISC_FEATURE (1)..(1): acetyl, ClAc, BrAc
  MISC_FEATURE (1)..(1): X is A or D and when X is A, then A is either cyclohexylalanine or D-alanine
  MOD_RES (19)..(19): AMIDATION
XGQEEKAGGVVSTGLIGGG

SEQ ID NO: 21 - protein, 17 aa, Artificial Sequence
  Completely synthesized peptide construct
  MISC_FEATURE (7)..(8): gamma aminobutyric acid (gaba) or 3 amino propanoic acid (apa)
  MOD_RES (17)..(17): AMIDATION
DGQEEKAVVSTGLIGGG

SEQ ID NO: 22 - protein, 16 aa, Artificial Sequence
  Completely synthesized peptide construct
  MISC_FEATURE (1)..(1): acetyl, ClAc, BrAc
  MISC_FEATURE (1)..(1): X is A or D and when X is A, then A is either cyclohexylalanine or D-alanine
  MISC_FEATURE (5)..(5): GG is replaced with gamma aminobutyric acid (gaba) or 3 amino propanoic acid (apa)
  MOD_RES (16)..(16): AMIDATION
XGQEAGVVSTGLIGGG

SEQ ID NO: 23 - protein, 15 aa, Artificial Sequence
  Completely synthesized peptide construct
  MISC_FEATURE (1)..(1): GG is replaced with gamma aminobutyric acid (gaba) or 3 amino propanoic acid (apa)
  MISC_FEATURE (1)..(1): acetyl, ClAc, BrAc
  MISC_FEATURE (1)..(1): X is A or E and when X is A, then A is either cyclohexylalanine or D-alanine
  MOD_RES (15)..(15): AMIDATION
XEKAGVVSTGLIGGG

SEQ ID NO: 24 - protein, 26 aa, Artificial Sequence
  Completely synthesized peptide construct
  MISC_FEATURE (1)..(1): acetyl, ClAc, BrAc
  MISC_FEATURE (1)..(1): X is A or N and when X is A, then A is either cyclohexylalanine or D-alanine
XGQEEKAGVVSTGLIQNGDWTFQTLV

SEQ ID NO: 25 - protein, 26 aa, Artificial Sequence
  Completely synthesized peptide construct
  MISC_FEATURE (1)..(1): acetyl, ClAc, BrAc
  MISC_FEATURE (1)..(1): X is A or N and when X is A, then A is either cyclohexylalanine or D-alanine
  MOD_RES (26)..(26): AMIDATION
XGQEEKAGVVSTGLIQDGDWTFQTLV

SEQ ID NO: 26 - protein, 27 aa, Artificial Sequence
  Completely synthesized peptide construct
  MISC_FEATURE (1)..(1): acetyl, ClAc, BrAc
  MISC_FEATURE (1)..(1): X is A or N and when X is A, then A is either cyclohexylalanine or D-alanine
  misc_feature (18)..(18): Xaa can be any naturally occurring amino acid
  MISC_FEATURE (19)..(19): hydrophilic aminobutanoic acid (aba)
  MISC_FEATURE (19)..(19): S or not present
  MOD_RES (19)..(19): AMIDATION
XGQEEKAGVVSTGLIQAXGDWTFQTLV

SEQ ID NO: 27 - protein, 22 aa, Artificial Sequence
  Completely synthesized peptide construct
  MISC_FEATURE (1)..(1): acetyl, ClAc, BrAc
  MISC_FEATURE (1)..(1): X is A or E and when X is A, then A is either cyclohexylalanine or D-alanine
  MOD_RES (22)..(22): AMIDATION
XKAGVVSTGLIQNGDWTFQTLV

SEQ ID NO: 28 - protein, 17 aa, Artificial Sequence
  Completely synthesized peptide construct
  MISC_FEATURE (1)..(1): acetyl, ClAc, BrAc
  MISC_FEATURE (1)..(1): X is A or G and when X is A, then A is either cyclohexylalanine or D-alanine
  MISC_FEATURE (10)..(11): gamma aminobutyric acid (gaba) or 3 amino propanoic acid (apa)
  MOD_RES (17)..(17): AMIDATION
XGGILGTSVVAKEEQGD

(19) United States
(12) Patent Application Publication
(10) Pub. No.: US 2007/0003542 A1
(43) Pub. Date: Jan. 4, 2007

(54) Methods for treating diseases or conditions with peptide constructs

(76) Inventors: Daniel Zimmerman, Bethesda, MD (US); Yupin Charoenvit, Silver Spring, MD (US); Kenneth Rosenthal, Akron, OH (US)

Correspondence Address: HAHN & VOIGHT PLLC, 1012 14TH STREET, NW, SUITE 620, WASHINGTON, DC, 20005, US

(21) Appl. No.: 10/502,328

(22) PCT Filed: Jan. 23, 2003
(86) PCT No.: PCT/US03/01816

§ 371 Date: May 19, 2005

Related U.S. Application Data
(60) Provisional application No. 60/350,032, filed on Jan. 23, 2002.
(60) Provisional application No. 60/349,982, filed on Jan. 23, 2002.
(60) Provisional application No. 60/349,983, filed on Jan. 23, 2002.

(51) Int. Cl. A61K 39/395; A61K 39/00

(52) U.S. Cl. 424/131.1; 424/185.1

(57) ABSTRACT

This invention relates to peptides directing a CD4 related T helper cell response wherein the peptides may be used as an adjuvant provided with an antigen or as an immunomodulatory agent without an antigen and compositions comprising modification of a fifteen-mer peptide sequence from the MHC IIβ chain at positions 135-149 known as Peptide G or a derivative of derG or other derivatives wherein the derivatives enhance the immune response of antigens and methods for treating cancer, autoimmune disease, transplant conditions, infectious conditions or allergies caused by foreign eukaryotic organisms, and infectious conditions or allergies caused by prokaryotic organisms or nonliving agents such as viruses, phages and prions with polypeptides.

### BACKGROUND OF THE INVENTION

1. Field of the Invention

This invention relates to peptides directing a CD4 related T helper cell response wherein the peptides may be used as an adjuvant provided with an antigen or as an immunomodulatory agent without an antigen. This invention further relates to compositions comprising modification of a fifteen-mer peptide sequence from the MHC IIβ chain at positions 135-149 known as Peptide G or a derivative of derG or other derivatives wherein the derivatives enhance the immune response of antigens. This invention further relates to methods for treating cancer, autoimmune disease, transplant conditions, infectious conditions or allergies caused by foreign eukaryotic organisms, and infectious conditions or allergies caused by prokaryotic organisms or non-living agents such as viruses, phages and prions with polypeptides as shown in SEQ ID NO.'s 1-28.

2. Description of the Related Art

A known class of immunologically active and diagnostic peptide constructs obtained by joining one or more T cell binding ligands with an antigenic peptide are L.E.A.P.S.™ (Ligand Epitope Antigen Presentation System) constructs described in U.S. Pat. No. 5,652,342 and U.S. Pat. No. 6,096,315, the entirety of which are incorporated herein. L.E.A.P.S.™ constructs are bi- or hetero-functional peptides having an antigenic peptide (Ag) linked to another peptide referred to as a T cell binding ligand (TCBL). This complex allows the presentation of antigen to occur at the same time as delivery of a costimulatory signal.

As described in the referenced documents, linking a T cell binding ligand to a peptide epitope alters the nature of the immune response (i.e., cell mediated (TH1)- or antibody (TH2) response). Based on this L.E.A.P.S.™ technology, specific classes of peptide constructs have been developed for immunological disorders such as HIV as shown in U.S. Pat. No. 6,093,400, U.S. Pat. No. 6,268,472, U.S. Pat. No. 6,103,239, U.S. Pat. No. 6,287,565, U.S. Pat. No. 6,111,068, U.S. Pat. No. 6,258,945, PCT/US98/20681, PCT/US 00/41647, PCT/US 00/41646, and PCT/US 01/16793, the disclosures of which are incorporated herein.

In some cases, antibodies derived from conjugated peptides (also referred to as a “peptide construct”) are better able to recognize native molecules than antibodies prepared using a conventional peptide-KLH conjugate. Moreover, antibodies induced by the conjugated peptide have a broader specificity recognizing the peptide epitope not only in the free linear peptide form but also in the native molecule. In contrast, peptides conjugated to KLH often fail to recognize the epitope in the native molecule. An exemplary of the T cell binding ligand portion of the above described peptide constructs is a modification of a portion of the MHC Class IIβ from residues 135-149 (“Peptide G”).

Peptide constructs were formed using Peptide G dissolve soon after preparation in saline (0.15 M NaCl, pH 7.4) or in water for injection (WFI) at a concentration of 0.5-2.0 mg/ml. HPLC methods applied to the peptide construct solutions maintained at temperatures of 2-8° C., i.e., refrigerated; or 18-25° C., i.e., room temperature; or 40° C., i.e., elevated, over periods of hours to days, and at pH values of from 7.4 down to 4.5 resulted in peptide constructs prone to deamination, particularly at higher pH's. Deamination was observed at the amino terminus and yielded either an isoaspartic or aspartic acid residue.

Accordingly, it would be highly desirable to provide specific amino acid substitutions in order, for example, to increase stability or biological half life by reducing sensitivity to various proteases, to alter or enhance binding to its preferred natural ligand, to provide specific binding sites or for the purpose of introducing a label, e.g., radioactive or fluorescent tagging. It is also well recognized by those skilled in the art that peptide mimetics which possess the same natural ligand may be useful. Pharmaceutical compositions based on these mimetics and variations thereof are also desirable.

Since the present invention contemplates peptides useful for directing a CD4 related T-helper cell response wherein the peptide may be used as an adjuvant provided with the antigen or as an immunomodulatory agent without the antigen, it would also be desirable to provide for potentially powerful adjuvants or immunomodulatory agents for preventing and/or treating diseases involving tumor antigens and/or self antigens such as cancers (e.g. prostate cancer, melanoma, colorectal cancer, lung cancer, breast cancer, kidney cancer, bone cancer, leukemia, adrenal cancer, ovarian cancer, cervical cancer, skin cancer, etc.), Alzheimer's dementia, ALS, transplantation disorders and autoimmune conditions such as diabetes, rheumatoid arthritis, lupus, MS, myocarditis as well as infectious diseases caused by viruses and their products (such as HSV, HBV, HCV, HPV), prions (CJD, vCJD, BSE, Scrapie) and infectious bacteria (prokaryotic organisms) such as Mycobacterium, Clostidium botulism, Salmonella, Staphylococcus, Streptococcus, Anthrax, etc. and also diseases or infections caused by non-self eukaryotic organisms or their products such as Lesihmania ascaris, flukes, worms, Plasmodium for malaria, and Amoeba for dysentery.

In particular, the present invention may be useful in the treatment of pathological responses involving unwanted T cell activation such as allergic diseases associated with particular MHC alleles suspected of having an autoimmune component. Other deleterious T cell-mediated responses including the destruction of foreign cells purposely introduced into the body as grafts or transplants from allogeneic hosts. For example, allograft rejection involves the interaction of host T cells with foreign MHC molecules. Quite often, a broad range of MHC alleles are involved in the response of the host to an allograft.

Another class of deleterious immune mediated response is autoimmune disease. Autoimmune disease results in loss of self-tolerance wherein the immune system attacks “self” tissue as if it were a foreign target. More than 30 autoimmune diseases are presently known including myasthenia gravis (MG), multiple sclerosis (MS), Rheumatoid arthritis, and Insulin Dependent Mellitus.

The present invention, therefore, provides peptides, which provide powerful stimulants for neutralizing and/or killing infected organisms and methods for using the compositions of the present invention as an adjuvant or immunomodulatory agent for patients at risk for or exposed to various diseases.

Further improvements and uses of peptide G and derG as an adjuvant, immunostimulator or immunomodulator when used with or without an antigen are described herein as well as other embodiments of the invention.

### SUMMARY OF THE INVENTION

The present invention is based, in part, on the discovery that the modified version of Peptide G (Asn Gly Gln Glu Glu Lys Ala Gly Val Val Ser Thr Gly Leu Ile—SEQ ID NO. 5) obtained by replacing Asn with Asp to form der G (Asp Gly Gln Glu Glu Lys Ala Gly Val Val Ser Thr Gly Leu Ile—SEQ ID NO. 7) has significantly more potent biological activity than the parent molecule. The peptides enhance the immune response, particularly the CD4 related (cell mediated) response, independent of being supplied as a conjugated peptides (L.E.A.P.S.™ constructs) as previously described. Isoaspartic acid is not used since it is not naturally found in proteins or encoded by the genetic code. Accordingly, the present invention enables the development of compositions useful as a pharmaceutical, adjuvant, immunostimulant or immunomodulator to activate the immune system wherein the compositions may be peptides, non-peptide mimetics or organic molecules selected from aliphatics, carbohydrates, heterocyclics, aromatics, substituted forms and mixtures thereof.

In accordance with a first embodiment of the present invention, there is provided a peptide comprising an amino acid sequence corresponding to the amino acid sequence of SEQ ID NO.'s 1-25 useful as an immunomodulator or adjuvant by directing a host to mount an enhanced Th1 response which may enhance a person's ability to respond to eliminate antigenic materials.

In accordance with another embodiment of the invention, there is provided a pharmacologically effective composition of the above mentioned peptide (SEQ ID NO.'s 1-28) supplied as an immunomodulator or immunoenhancing agent without concomitant administration of antigen.

In accordance with yet another embodiment of the invention, the peptide (any one of SEQ ID NO. 5-28) is supplied as a parenteral (by injection), transdermal (through the skin), oral, nasal (administered by an aerosol mist), rectal (suppository) or by other body orifices (eyes, urogenital, ear) in conjunction with one or more of other antigens, adjuvants, stabilizers or excipents.

Still yet another embodiment of the present invention is a peptide as set forth above as an immunogen for the production of antibodies. In one embodiment, there is provided an antibody produced in such an application. In another embodiment, the antibody is labeled. In yet another embodiment, the antibody is bound to a solid support. In yet a further embodiment, the antibody is monoclonal. In still yet another embodiment of the present inventive subject matter is an oligonucleotide primer useful for amplification of DNA, the oligonucleotide primer designed on the basis of the DNA sequence of any one of SEQ ID NO.'s 5-28. The embodiments may also comprise an amino acid sequence substantially the same as the amino acid sequence set forth in SEQ ID NO.'s 1-4 or is at least 70% similar to all or a part thereof. Accordingly, the present embodiment of the present invention can be directed to a substantially similar isolated or recombinant polypeptide or a derivative, homologue or analogue thereof.

Another embodiment of the present invention is directed to a pharmaceutical composition for treating a disease requiring mediation of a CD4 related T-helper cell response, comprising a therapeutically effective amount of an amino acid sequence as shown in SEQ ID NO.'s 1-28 and a pharmaceutically acceptable carrier.

Additional embodiments include:

a method for treating cancer, autoimmune, or transplant conditions, comprising administering a polypeptide as shown in SEQ ID NO.'s 1-28 to an animal in need thereof;

a method for determining an immunomodulatory agent as a prophylactic or a therapeutic with applications in cancers, autoimmune and transplant rejection conditions, comprising the steps of evaluating SEQ ID NO.'s 1-28 using Microarray technology; a method for treating infectious conditions or allergies caused by foreign (i.e. not self) eukaryotic organisms, comprising administering a polypeptide as shown in SEQ ID NO.'s 1-28 to an animal in need thereof;

a method for determining an immunomodulatory agent as a prophylactic or a therapeutic for treating infectious conditions or allergies caused by foreign (i.e. not self) eukaryotic organisms, comprising the steps of evaluating SEQ ID NO.'s 1-28 using Microarray technology;

a method for treating infectious conditions or allergies caused by parasitic organisms, comprising administering a polypeptide as shown in SEQ ID NO.'s 1-28 to an animal in need thereof;

a method for treating infectious conditions caused by prokaryotic organisms or non-living agents such as bacterial viruses, phages and prions, comprising administering a polypeptide as shown in SEQ ID NO.'s 1-28 to an animal in need thereof; and

a method for determining an immunomodulatory agent as a prophylactic or a therapeutic with applications in treating disease or infectious conditions caused by prokaryotic organisms or non-living agents such as bacterial viruses, phages and prions, comprising the steps of evaluating SEQ ID NO.'s 1-28 using Microarray technology.

One of ordinary skill in the art will appreciate that other aspects of this invention will become apparent upon reference to the attached figures and the following detailed description.

### BRIEF DESCRIPTION OF THE DRAWINGS

The following description makes reference to the text in the accompanying drawings to provide a better understanding of the embodiments of the present invention, whereby:

FIG. 1 is a graph showing immunization and challenge of L.E.A.P.S.™ constructs of the TRP2₁₈₀₋₁₈₈ is peptide with peptide derG (LEAPS 2) or peptide J (LEAPS 1) or derG plus Peptide J as a TCBL.

FIG. 2 is a graph measuring IFN-γ production in spleen cell cultures obtained from outbred CD-1 mice.

FIG. 3 is a graph of a zosteriform spread of lesions and timing of CEL-1000 administration in a HSV Zosteriform murine model.

FIG. 4 is a graph of survival and timing of CEL-1000 administration in a Zosteriform murine model.

FIG. 5 is a graph of a comparison of route of CEL-1000 administration 2 weeks prior to challenge.

### DETAILED DESCRIPTION OF THE INVENTION AND PREFERRED EMBODIMENTS

Background

L.E.A.P.S.™ constructs include an antigenic peptide (Ag) linked to another peptide referred to as a T cell binding ligand (TCBL). This complex may allow the presentation of antigen to occur at the same time as delivering a costimulatory signal. Adjuvants such as alum, MPL S/E™ (“Lipid A”), Muramyl Dipeptide (“MDP”) or other saponin derivatives, as well as small molecular weight entities such as PLG and QS21 have been used with antigens to improve the immune response.

Cytokines and immunomodulators such as IL-2, IL-10, IL-12, GM-CSF, Flt-3L, CD40L, or Ox40 and cytokine genes have also been used as pretreatment or been simultaneously administered in combination with a mixture of antigens, fusion protein or separate genes. Notably, cytokines greater than or equal to 10,000 kDa are much larger in size than Lipid A and MDP while QS21 and other saponin derivatives are lipid soluble and in between in size.

Several types of TCBL's have been evaluated. Of particular interest are peptides J and G, and an improved version, derG; wherein peptide J is a short fragment from β₂-microglobulin (a.a. 38-50) and derG is a modified fragment from MHC II β-chain (a.a. 135-149). Conjugates of these appear to activate different sub-sets of T cells. Based on site directed mutagenesis studies of MHC II β-chain and/or peptide competition studies, peptide G presumably binds to CD4, a T cell co-stimulator molecule. Peptide J's ligand is less well defined, but based upon monoclonal antibody binding experiments, a portion of the peptide J region is exposed when complexed to the MHC I antigen complex on APC. Conjugates of these peptides appear to activate different sub-sets of T cells.

TABLE 1
TCBL Peptides to be used SBIR Section D

| | | |
|---|---|---|
| Peptide | Sequence | Ref |

| | | |
|---|---|---|
| G | NGQEEKAGVVSTGLIGGG | Zimmerman et al. 1996 |
| derG | DGQEEKAGVVSTGLIGGG | Zimmerman et al. 2001 |
| J | DLLKNGERIEKVEGGG | Zimmerman et al. 1996 |

In a murine system, immuno-stimulation of an antigen specific Th1 immune response and subsequent inhibition of such activation results in cell death where the CD4 binding site interacts with peptides having a homologous sequence (Peptide G) from I-A β^{k}. However, other researchers report that immuno-stimulation of an antigen specific Th1 immune response enhanced antigen specific in vitro stimulation of IFN-γ (Shen et al. “Regulation of T cell immunity and tolerance in vivo by CD4”, Int Immunol. 1998 10:247-57 and Shen et al. “Peptides corresponding to CD4-interacting regions of murine MHC class II molecules modulate immune responses of CD4+ T lymphocytes in vitro and in vivo”, J. Immunol. 1996 157:87-100). Clayberger et al. further discloses that peptides from this same region inhibit in vitro CTL proliferation of fresh PBL and allogenic responses (“Peptides corresponding to the CD8 and CD4 binding domains of HLA molecules block T lymphocyte immune responses in vitro”, J Immunol 1994 153:946-51). Notably, Gilfillan et al. (“Selection and function of CD4+ T lymphocytes in transgenic mice expressing mutant MHC class II molecules deficient in their interaction with CD4”, J. Immunol. 1998 161:6629-37) discloses that neutralization of the amino terminus occurs when using a propionylated peptide (See also Shen et al. “Peptides corresponding to CD4-interacting regions of murine MHC class II molecules modulate immune responses of CD4+ T lymphocytes in vitro and in vivo”, J Immunol. 1996 157:87-100).

The human counterpart of the peptides are used in L.E.A.P.S.™ construct as TCBL portions of the construct. In particular, the peptides facilitate antigen presentation albeit at a lower concentration and with less frequent dosings than the murine experiments. Notably, human MHC II β₁₃₅₋₁₄₉ based TCBL's bind to murine CD4 as well as human CD4 (Cammarota et al., “identification of a CD4 binding site on the beta 2 domain of HLA-DR molecules”, Nature 1992; 356; pp. 799-801). Additionally, a second site on the MHC binds to another CD4 epitope. While both sites are important in development and maturation of a complete response, binding events occur sequentially because both MHCII CD4 interactions cannot occur at the same time (Yelon et al., “Alterations in CD4-binding regions of the MHC class II molecule I-Ek do not impede CD4+T cell development”; J. Immunol; 1999; 162; pp. 1348-1358; Yelon et al., “Alterations in CD4 dependence accompany T cell development and differentiation”, Int. Immunol. 1996 8:1077-90; Mostaghel et al., “Coreceptor—independent T cell activation in mice expressing MHC class II molecules mutated in the CD 4 binding domain”, J Immunol 1998 161:6559-66; and Riberdy et al., “Disruption of the CD4 major histocompatibility complex class II interaction blocks the development of CD4(+) T cells in vivo”, Proc. Natl. Acad. Sci. USA 1998 95:4493-8).

derG Protective Effect with Tumor System

Confirmation that derG may possess its own immunostimulatory or adjuvant properties for a tumor system are shown in Example 1 of the Examples. Many cancers express similar arrays of antigens and mutations wherein the vaccines are based on shared antigens. Therefore, by vaccinating with allogeneic tumors from the same cancer, a T-cell response may be stimulated by antigens shared between the vaccine and the patient's own tumor.

Advantageously, allogenicity obviates the need for time-prohibitive growing of syngeneic tumor for vaccine development. Further still, the host versus graft induced reaction encountered by the vaccine may actually promote antigen processing and thereby improve efficacy of the vaccine. Proof of principle studies based on an adaptation of the murine B16 melanoma model demonstrate this theory.

In particular, C57/b16 (H2^{b}) mice are vaccinated with the allogeneic melanoma K1735 (H2^{k}) at least twice on weekly intervals and then challenged with syngeneic B16 (H2^{b}) tumors. Both the therapeutic and prophylactic forms in the model demonstrate significant protection. Investigation of the immune response elicited by allo-vaccination also revealed that cytotoxic T-lymphocytes (CTL) specific for the syngeneic tumor develop after vaccination with an allogeneic tumor. This is clear evidence of cross-priming and the existence of shared antigens.

Immunization of L.E.A.P.S.™ constructs of the TRP2₁₈₀₋₁₈₈ peptide with peptide derG or J and use of a mixture of derG or J (TCBL pool) established immunostimulatory or adjuvant activity of derG. The repeated experiments produced consistent results. However, the allogeneic vaccine only showed minor efficacy. Moreover, no protection was seen when J-TRP2₁₈₀₋₁₈₈ (LEAPS 1) or the TCBL pool (derG+J) were used in isolation. Notably, J-TRP2₁₈₀₋₁₈₈ (LEAPS 1) in combination with K1735 showed no protection. The results suggest that the key species is the derG peptide as a conjugate or separate entity.

Although the TCBL pool was a mixture of both derG (TCBL2) and J (TCBL), no efficacy was detected in the absence of allogeneic cells. On the other hand, a combination of cells resulted in a protection of 40% and 20% for both experiments. Since the TCBL pool was a mixture, it is difficult to assess which TCBL is active. When derG was administered in combination with cells, protection was seen at a level similar to that of TCBL (30% and 40%). Notably, derG-TRP2₁₈₀₋₁₈₈ on its own demonstrated an efficacy of 20%.

These data suggest that derG may be the active principle in the compounds tested. This peptide is present in both derG-TRP2 J-TRP2₁₈₀₋₁₈₈ (LEAPS 2) and the mixture of TCBL (J+derG), giving a similar level of protection in both cases. Protection is only manifest when allogeneic tumor cells are present, suggesting that derG enhances specific T-cell immunity.

Given that CTL are likely to be a major protective element in these systems, measurement of killing activity using Cr⁵¹ is a useful bio-marker of derG activity. Clearly, allogeneic vaccination gives rise to a CTL generation capable of killing syngeneic tumor. Moreover, derG effects alone or in combination with antigen (allogeneic tumor cell vaccination) could not have occurred by chance. This is especially important when considering the differences from the parental peptide G which was less active.

Another method capable for determining the biological activity and mechanisms of derG is utilization of high density oligonucleotide microarrays as disclosed in Watanabe et al. Proc. Nat. Acad. Sci. 98:6577 (2001). Similar to evaluating the effect of Ginko biloba on mice brain tissue, expression and transcription of genes showing novel profiles of cytokines, cellular differentiation or activation antigens from RNA extracted from the lymphoid or the infiltrating tissues or cells of test animals. Once a particular gene(s) and its encoded protein(s) are identified, commercially available assays can be used to monitor the amount of protein induced after treatment with derG or a related agent.

Malaria L.E.A.P.S.™ Constructs and derG TCBL

Evaluation of the TCBL's biological activity when used as an additive or as an immunostimulant showed desirable results in malaria protection assays as shown in Examples 2 and 3 of the Examples. An antigen and peptide G MHC-II β₁₃₄₋₁₃₈ conjugated into a single species demonstrated substantial improvement in protective efficacy.

A substantial improvement in protection from infection was also seen in groups receiving the improved TCBL derG whether conjugated or alone. Based upon the earlier results with the W and G peptide, IFN-γ levels in the serum pools were evaluated. However no protection was seen in the Balb/C strain. FACS analysis measuring the presence of IFN-γ⁺/CD4⁺ cells isolated from liver (the major site for infection) by perfusion and from spleen cell populations further showed increased levels of IFN-γ especially for liver cells.

Another experiment similar to the first was conducted except only a single strain of mice (A/J) was used with decreasing doses of the derG in order to evaluate potency. Again the results were consistent. Still another experiment evaluated the protective efficacy of derG in three other mouse strains: BALB/c (H2d), C3H/HeJ (H2K) and hybrid CAF1 (A/J×BALB/c) wherein the results indicate that derG protects C3H/HeJ but not BALB/c and hybrid CAF1 (A/J×BALB/c).

HSV-1 and Scarification Zosteriform Spread Model of Infection

A Zosteriform Spread model of HSV infection evaluated the efficacy of antiviral drugs and vaccines wherein an improved animal model discriminates between neuroinvasive and non-neuroinvasive viruses. The scarification (abrasion)-zosteriform spread model of infection clearly demonstrated that CEL-1000 (derG) protects A/J mice from lethal HSV-1 challenge.

Disease progression was monitored and the progression and the severity of the lesions scored from 0 to 7 (death). In some experiments modifications in the timing, route of administration and mouse strain were evaluated for efficacy of CEL-1000. The model also evaluated the timing of administration of CEL-1000 using a single dose of 25 μg. In particular, a delay in onset of symptoms such as reduced morbidity, zosteriform spread and mortality was observed. Survival was observed for A/J mice treated with a single dose of CEL-1000 (25 μg) emulsified in Seppic ISA-51 on day 28, 14, 7 prior to challenge with HSV-1.

Surprisingly, intramuscular administration is more effective. Complete (100%) protection was achieved when CEL-1000 was administered intramuscularly 2 weeks prior to challenge. In addition, the dose was delivered without adjuvant in aqueous solution at the same concentration as the other treatments thereby suggesting that adjuvant is not necessary.

Similar protective effects of CEL-1000 were seen in other strains (both C57BL6, another inbred strain with a different MHC background and CD1 an outbred strain). Clearly, CEL-1000 (derG) prolonged survival against HSV-1 challenge.

Peptide Constructs

For the peptides disclosed in this application, the amino acid sequences thereof, are set forth by the single letter and three-letter identification symbols as follows:

| | Three-letter | One-letter |
|---|---|---|
| Amino Acid | abbreviation | symbol |
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

SEQ ID NO.'S 1-28 can be modified to increase stability or biological half life by reducing sensitivity to various proteases, to alter or enhance binding to its preferred natural ligand, to provide specific binding sites or for the purpose of introducing a label such as radioactive or fluorescent tagging. It is also well recognized by those skilled in the art that peptide mimetics which possess the same natural ligand may be useful.

Small molecules can also be designed by one of ordinary skill in the art to bind to the same sites as SEQ ID NO.'s 1-4 displacing the polypeptides of SEQ ID NO.'s 1-4 due to higher binding affinity. The molecules can be chosen from the following classes of molecules including but not limited to aliphatics, carbohydrates, heterocyclics, aromatics or mixtures thereof. Preferred mimetics will include atoms at positions similar to those of the erythropoietin (EPO) receptor contacting atoms of an EPO mimetic such as EMPI described in U.S. Pat. No. 5,835,382, the entirety of which is incorporated herein by reference. Even more preferred mimetics will be structurally similar to the polypeptides of SEQ ID NO.'s 1-28.

Methods known by those skilled in the art in the design of small molecule and polypeptide mimetics employ a computer-based methods for identifying compounds having a desired structure. More specifically, the invention uses the three-dimensional coordinates of a subset of the atoms in the peptide when the peptide is co-crystallized with a portion of the T-cell binding receptor to determine peptide and non-peptide mimetic candidates by means of computer methods.

These computer-based methods fall into two broad classes: database methods and de novo design methods. In database methods the compound of interest is compared to all compounds present in a database of chemical structures and compounds whose structure is in some way similar to the compound of interest are identified. The structures in the database are based on either experimental data generated by NMR or x-ray crystallography, or modeled three-dimensional structures based on two-dimensional (i.e., sequence) data. In de novo design methods, models of compounds whose structure is in some way similar to the compound of interest are generated by a computer program using information derived from known structures, e.g., data generated by x-ray crystallography and/or theoretical rules. Such design methods can build a compound having a desired structure in either an atom-by-atom manner or by assembling stored small molecular fragments. The success of database and de novo methods in identifying compounds with activities similar to the compound of interest depends on the identification of the functionally relevant portion of the compound of interest.

For drugs, the functionally relevant portion is referred to a pharmacophore. A pharmacophore is an arrangement of structural features and functional groups important for biological activity. Not all identified compounds having the desired pharmacophore will act as an TCBL mimetic. The actual activity is finally determined by measuring the activity of the compound in relevant biological assays. However, the methods of the invention are extremely valuable because they can be used to greatly reduce the number of compounds which must be tested to identify an actual mimetic.

The peptide fragments contemplated by the present invention are:

MHC II β₁₃₈₋₁₅₂

SEQ ID NO. 1

| | |
|---|---|
| EKAGVVSTGLIQNGD | (Cammarota et al 1992) |

MHC II β₁₃₄₋₁₄₈

SEQ ID NO. 2

| | |
|---|---|
| NGQEEKAGVVSTGLI | (Cammarota et al 1992) |

MHC II β₁₃₇₋₁₄₇

SEQ ID NO. 3

| | |
|---|---|
| EETVGVSQLEV | (Konig et al 1992) |

I-Ad β₁₃₄₋₁₄₈

SEQ ID NO. 4

| | |
|---|---|
| NGQEETVGVSSTQLI | (Shen et al 1996) |

Below are various categories of modifications intended to improve the biological properties of the above sequences.

Group 1

Use of amidation or esterification at carboxyl terminus to reduce sensitivity to carboxylpeptidases of peptide(s).

NGQEEKAGVVSTGLIamide
EETVGVSQLEVamide

Group 2

Adding several amino acids for example GGG, but not restricted to same, at carboxyl end to facilitate conjugation and spacing in certain conjugates.

| | |
|---|---|
| NGQEEKAGVVSTGLIGGGamide | SEQ ID NO. 5 |
| EETVGVSQLEVGGGamide | SEQ ID NO. 6 |

Group 3

Changing the amino terminus amino acid to a more stable one N to D or change amino terminus to aspartic acid from unstable asparagines when adjacent to Glycine.

| | | |
|---|---|---|
| derG | DGQEEKAGVVSTGLIGGGamide | SEQ ID NO. 7 |
| | DGQEEKAGVVSTGLI | SEQ ID NO. 18 |

Group 4

Using acetylated, propionylated, bromo or Chloro of amino terminus amino acid to reduce proteolysis in vivo and thus increase half-life, as shown in following peptides:

| | |
|---|---|
| ClAcDGQEEKAGVVSTGLIGGG-amide | |
| BrAcDGQEEKAGVVSTGLIGGG-amide | |
| ClAcGQEEKAGVVSTGLIGGG-amide | SEQ ID NO. 8 |
| BrAcGQEEKAGVVSTGLIGGG-amide | |
| BrAcEETVGVSQLEVGGGamide | |
| ClAcEETVGVSQLEVGGGamide | |
| AcetylDGQEEKAGVVSTGLIGGG-amide | |
| AcetylEETVGVSQLEVGGGamide | |

wherein ClAc represents chloroacetic acid or BrAc for Bromoacetic acid as shown in U.S. Pat. No. 5,066,716 and incorporated herein by reference and acetyl represents a acetylated group added at amino terminus.
Group 5

Using amino acid analogues (see U.S. Pat. No. 5,736,412) cyclohexylalanine represented by B to reduce potential for rapid proteolysis.

BGQEEKAGVVSTGLIGGGamide
BEETVGVSQLEVGGGamide

Or using D-amino acids especially D-Alanine represented by Z, (U.S. Pat. No. 5,736,412) to reduce potential for rapid proteolysis

ZGQEEKAGVVSTGLIGGGamide
ZEETVGVSQLEVGGGamide

Group 6

Using substitutions at sites interacting with CD4 that increase binding to CD4 molecules identified in the attached sequence listings. Conserved substitutions with a similar type of amino acid are listed as follows from the groupings. If within the same category but on a different level they are not considered as a conserved substitution.

| | | |
|---|---|---|
| Non Polar | | G, A, (P) |
| | | V, L, I |
| Polar | Neutral | C, M, S, T |
| | | N, Q |
| | Acidic | D, E |
| | Basic | K, R (H) |
| Aromatic | | F, W, Y, (H) |

such as E137D and/or A140G (V, L, I) and or V142I, L (or G, A) wherein the substitutions in parenthesis are less similar. Additional substitutions are shown below.

```
NGQEEKAGVVSTGLIGGGamide
NGQEEKAGVVSTGLIGGGamide
NGQEEKAGVVSTGLIGGGamide
NGQEEKAGVVSTGLIGGGamide
NGQEEKAGVVSTGLIGGGamide
NGQEEKAGVVSTGLIGGGamide
      EETVGVSQLEVGGGamide
      EETVGVSQLEVGGGamide
      EETVGVSQLEVGGGamide
      EETVGVSQLEVGGGamide
      EETVGVSQLEVGGGamide
      EETVGVSQLEVGGGamide
```

Group 7

Using substitutions at sites not interacting with CD4 especially if protease-sensitive especially arginine, lysine or cysteine and making use of other conserved substitution for Lysine by use of analogues such as substituted episoln amino (methyl, alkyl) Lysine or hydroxyl-Leucine or Leucine.

| | |
|---|---|
| DGQEEKAGVVSTGLIGGGamide | SEQ ID NO. 9 |
| DGQEEFAGVVSTGLIGGGamide | SEQ ID NO. 10 |
| ClAcGQEEKAGVVSTGLI-amide | |
| BrAcGQEEKAGVVSTGLI-amide | |
| AcetylGQEEKAGVVSTGLI-amide | |
| ZEETVGVSQLEVamide | |
| BEETVGVSQLEVamide | |
| ClAcEETVGVSQLEVamide | |
| BrAcEETVGVSQLEVamide | |

Group 8

Using shortened forms of above SEQ ID NO.s 1-10.

| | |
|---|---|
| BrAcETVGVSQLEVamide | SEQ ID NO. 11 |
| BETVGVSQLEVamide | SEQ ID NO. 12 |
| BETVGVSQLEamide | SEQ ID NO. 13 |
| BETVGVSQLamide | SEQ ID NO. 14 |
| BETVGVSQamide | SEQ ID NO. 15 |
| BETVGVSamide | SEQ ID NO. 16 |
| BETVGVamide | SEQ ID NO. 17 |

Furthermore, CLIP or antigenic peptides each coil in the antigenic peptide binding site as polyprolyl type II (PPII) helices with the amino acid repeat frequency per turn of 3.0 amino acids, whereas it is 3.2 for alpha helices. Examination of the sequence of derG shows that only 1 amino acid is found between two amino acids identified by site directed mutagenesis studies as critical (if we use the identified first V as the critical “V” see below:

| | |
|---|---|
| NGQEEKAGVVSTGLI | SEQ ID NO. 2 |

An increase by one amino acid to allow for presentation of “A” and “V” on the same face insertion of one “G” better presents the residues in the same plane.

| | |
|---|---|
| DGQEEKAGGVVSTGLIGGGamide | SEQ ID NO. 20 |

Although Humphries et al. (2000 Vaccine 18:2693-7) discloses a 5-aminopentanoic acid replacing four amino acids (LMRK) in their peptide and adds four methylene bridges (—HN—CH₂—CH₂—CH₂—CH₂—CO₂), the distal C for the amino function is required. Not the first or a C in the amino acid. Useful replacement of two amino acids for spacers include gamma aminobutyric acid (gaba) or (HN—CH—CH₂—CH₂—CO₂) and 3 amino propanoic acid (apa) or (HN—CH₂—CH₂—CO₂) replacing 3 or 2 amino acids.

Therefore, GG should be replaced by gaba or apa although other similar substitutions would be within the scope of this inventions as follows:

| | |
|---|---|
| DGQEEKAapaVVSTGLIGGGamide | SEQ ID NO. 21 |
| DGQEEKAgabaVVSTGLIGGGamide | |

Examples of other sites where this substitution is illustrated are as follows:

| | |
|---|---|
| DGQEapaAGVVSTGLIGGGamide | SEQ ID NO. 22 |
| DGQEapaAGGVVSTGLIGGGamide | |
| DGQEgabaAGVVSTGLIGGGamide | |
| DGQEgabaAGGVVSTGLIGGGamide | |
| paEEKAGVVSTGLIGGGamide | SEQ ID NO. 23 |
| apaEEKAGGVVSTGLIGGGamide | |
| abaEEKAGVVSTGLIGGGamide | |
| gabaEEKAGGVVSTGLIGGGamide | |

Another critical point for contact with CD4 is an extended sequence to encompass more of the molecule as follows:

NGQEEKAGVVSTGLIqngdwtfqtlv.

The residues shown in lower case represent highly variable sites. For example, the isoleucine at 143 and the leucine at 159 in contact with the CD4 phenylalanine as the 43 residue. Then the peptide could be used as the aspartic acid form as follows:

DGQEEKAGVVSTGLI qngdwtfqtv amide

or the amino blocked Ac, Pr, ClAc, or BrAc forms:

AcDGQEEKAGVVSTGLIqngdwtfqtlv amide
PrDGQEEKAGVVSTGLIqngdwtfqtlv amide
ClAcDGQEEKAGVVSTGLIqngdwtfqtlv amide
ClAcDGQEEKAGVVSTGLIqngdwtfqtlv amide.

Since the extended form has the NG sequence which has a tendency to deamidate, a more stable form may have the substitution to DG of:

| | |
|---|---|
| AcDGQEEKAGVVSTGLIqDgdwtfqtlv amide | SEQ ID NO. 25 |
| PrDGQEEKAGVVSTGLIqDgdwtfqtlv amide | |
| ClAcDGQEEKAGVVSTGLIqDgdwtfqtlv amide | |
| ClAcDGQEEKAGVVSTGLIqDgdwtfqtlv amide. | |

The less hydrophilic α-aminobutanoic acid (Aba) or S is also contemplated:

SEQ ID NO. 26

AcDGQEEKAGVVSTGLIqAAbagdwtfqtlv amide
PrDGQEEKAGVVSTGLIqAbagdwtfqtlv amide
ClAcDGQEEKAGVVSTGLIqAbagdwtfqflv amide
ClAcDGQEEKAGVVSTGLIqAbagdwtfqtlv amide
AcDGQEEKAGVVSTGLIqAbagdwtfqtlv amide
PrDGQEEKAGVVSTGLIqAbagdwtfqtlv amide
ClAcDGQEEKAGVVSTGLIqAbagdwtfqtlv amide
ClAcDGQEEKAGVVSTGLIqsgdwtfqtlv amide.

To shorten to reduce cost and size at the amino terminus till the first critical E137 the following constructs may be provided:

| | |
|---|---|
| AcEKAGVVSTGLIqngdwtfqtlvamide | SEQ ID NO. 27 |
| AcEKAGGVVSTGLIqngdwtfqtlvamide | |
| PrEKAGVVSTGLIqngdwtfqtlvamide | |
| PrEKAGGVVSTGLIqngdwtfqtlvamide | |
| ClAcEKAGVVSTGLIqngdwtfqtlvamide | |
| ClAcEKAGGVVSTGLIqngdwtfqtlvamide | |
| BrAcEKAGVVSTGLIqngdwtfqtlvamide | |
| BrAcEKAGGVVSTGLIqngdwtfqtlvamide. | |

The fragments ava, gaba (or apa) can be substituted for non contact residues in the previous regions and the VSTGL and QNGDWTFQT segments in the extended form. The position B (or D, ClAc, BRAc) at the amino terminus can be substituted for epsilon amino (methyl alkyl) lysine (Kea), hydroxyl-leucine (Loh) or isoleucine (I) for K protease sensitive sites. Phenylalanine (F) can be substituted for the more labile trytophane W and can also be substituted for the contact residues at E137, A140, V142I, L (or G, A).

The reverse order of amino acids for derG would be as follows can also be contemplated by the present invention:

| | |
|---|---|
| AcGGGILGTSVVAKEEQGDamide | SEQ ID NO. 28 |

including the D isomer form:

AcGGGILGTSVVAKEEQGDamide
AcILGTSVVAKEEQGDamide

and successively and sequentially eliminated amino acids down to

AcVAKEamide

The two inverso forms also could incorporate the previous substitutions of “gaba” or “apa”.

The present invention also includes combinations of two or more of the above improvements group 1 carboxyl terminus amide, group 2 carboxyl terminus extension of GGG, group 3 amino terminus modification (B or Z or BrAc or ClAc or Ac), group 4 binding site conserved substitution internal and Group 5 protease sensitive K substitution.

Use of peptide mimetics based upon x-ray crystallographic or other studies that bind to the same CD4 site in a similar manner as the derG peptide's E137, V140 and V(S) 142/3 and compete with derivatives described above of derG therefore for biological effect.

In addition to the variations in the amino acids, it is also recognized that the amino acids at the N-terminal and C-terminal may be present as the free acid (amino or carboxyl groups) or as the salts, esters, ethers, or amides thereof may increase stability and biological half-life of the immunomodulant or adjuvant peptide, derG. In particular amide end groups at the C-terminal and acetylation, e.g., myristyl, etc. at the N- or C-terminal, are often useful without effecting the immunological properties of the peptide.

The peptides and the constituent components thereof can be prepared by conventional processes for synthesizing proteins, for example solid phase peptide synthesis described by Merrifield, R. B., 1963 (J. of Am. Chem. Soc., 85:2149-2154). It is also within the scope of the invention and within the skill in the art to produce the novel peptide constructs of this invention or the peptide components thereof by genetic engineering technology.

The administration of the peptide of this invention may be carried out alone or in conjunction with other therapies. Examples of other therapies which may be used in conjunction with the peptide constructs of this invention include, in the case of treatments (prophylactic or therapeutic) for infection by HIV, for example, protease inhibitors, reverse transcriptase inhibitors and the like.

The peptide constructs of this invention may be used as a component of an immunomodulatory composition, together with one or more pharmaceutically acceptable carriers or adjuvants, either prophylactically or therapeutically. When provided for use prophylactically, the immunomodulatory composition is provided in advance of any evidence of infection or disease.

While it is possible for the immunogenic peptide construct to be administered in a pure or substantially pure form, it is preferable to present it as a pharmaceutical composition, formulation or preparation.

The formulations of the present invention, both for clinical and for human use, comprise a conjugated peptide as described above, together with one or more pharmaceutically acceptable carriers and, optionally, other therapeutic ingredients, especially therapeutic immunological adjuvants. The carrier(s) must be “acceptable” in the sense of being compatible with the other ingredients of the formulation and not deleterious to the recipient thereof.

In general, the formulations are prepared by uniformly and intimately bringing into association the active ingredient with liquid carriers or finely divided solid carriers or both, and then, if necessary, bringing the product into the desired formulation. The term “pharmaceutically acceptable carrier” as used herein refers to any carrier, diluent, excipient, suspending agent, lubricating agent, adjuvant, vehicle, delivery system, emulsifier, disintegrant, absorbant, preservative, surfactant, colorant, flavorant, or sweetener. The formulations may conveniently be presented in unit dosage form and may be prepared by any method well-known in the pharmaceutical art.

Possible formulations suitable for intravenous, intramuscular, subcutaneous, or intraperitoneal, nasal, administration comprise sterile aqueous solutions of the active ingredient(s) with solutions are preferably isotonic with the recipient's blood. The compounds of the present invention may also be administered orally, parenterally, by inhalation spray, topically, rectally, buccally, vaginally or via an implanted reservoir in dosage formulations containing conventional non-toxic pharmaceutically-acceptable carriers, adjuvants and vehicles. The term parenteral as used herein includes subcutaneous, intravenous, intramuscular, intraperitoneally, intrathecally, intraventricularly, intrasternal and intracranial injection or infusion techniques.

Such formulations may be conveniently prepared by dissolving solid active ingredient in water containing physiologically compatible substances such as sodium chloride (e.g. 0.1-2.0M), glycine, and the like, and having a buffered pH compatible with physiological conditions to produce an aqueous solution, and rendering the solution sterile. These may be present in unit or multi-dose containers, for example, sealed ampoules or vials.

For oral administration, the compounds of the present invention may be provided in any suitable dosage form known in the art. For example, the compositions may be incorporated into tablets, powders, granules, beads, chewable lozenges, capsules, liquids, aqueous suspensions or solutions, or similar dosage forms, using conventional equipment and techniques known in the art. Tablet dosage forms are preferred. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. Oral preparations may further be combined with typical carriers, such as talc, magnesium stearate, crystalline cellulose, methyl cellulose, carboxymethyl cellulose, glycerin, sodium alginate or gum arabic among others.

When preparing dosage form incorporating the compositions of the invention, the compounds may also be blended with conventional excipients such as binders, including gelatin, pregelatinized starch, and the like; lubricants, such as hydrogenated vegetable oil, stearic acid, and the like; diluents, such as lactose, mannose, and sucrose; disintegrants, such as carboxymethylcellulose and sodium starch glycolate; suspending agents, such as povidone, polyvinyl alcohol, and the like; absorbants, such as silicon dioxide; preservatives, such as methylparaben, propylparaben, and sodium benzoate; surfactants, such as sodium lauryl sulfate, polysorbate 80, and the like; colorants such as F.D. & C. dyes and lakes; flavorants; and sweeteners.

The formulations of the present invention may further incorporate a stabilizer. Illustrative stabilizers include polyethylene glycol, proteins, saccharides, amino acids, inorganic acids, and organic acids which may be used either on their own or as admixtures. These stabilizers, when used, are preferably incorporated in an amount of about 0.1 to about 10,000 parts by weight per part by weight of immunogen. If two or more stabilizers are to be used, their total amount is preferably within the range specified above. These stabilizers are used in aqueous solutions at the appropriate concentration and pH. The specific osmotic pressure of such aqueous solutions is generally in the range of about 0.1 to about 3.0 osmoles, preferably in the range of about 0.8 to about 1.2. The pH of the aqueous solution is adjusted to be within the range of about 5.0 to about 9.0, preferably within the range of 6-8. In formulating the immunostimulatory or adjuvant peptide of the present invention, an anti-adsorption agent may be used.

The compounds of the present invention may also be administered in the form of sterile injectable preparations, for example, as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. Among the acceptable vehicles and solvents that may be employed are water, Ringer's solution and isotonic sodium chloride solution. In addition, sterile, fixed oils are conventionally employed as solvents or suspending mediums. For this purpose, any bland non-toxic, fixed oil may be employed including synthetic mono- or di-glycerides. Fatty acids such as oleic acid and its glyceride derivatives, including olive oil and castor oil, especially in their polyoxyethylated versions, are useful in the preparation of injectables. These oil solutions or suspensions may also contain long-chain alcohol diluents or dispersants.

The compounds of this invention may also be administered rectally in the form of suppositories. These compositions can be prepared by mixing the drug with a suitable non-irritating excipient which is solid at room temperature, but liquid at rectal temperature and, therefore, will melt in the rectum to release the drug. Such materials include cocoa butter, beeswax and polyethylene glycols.

The compounds of this invention may also be administered topically, especially when the conditions addressed for treatment involve areas or organs readily accessible by topical application, including neurological disorders of the eye, the skin, or oral, nasal, vaginal, mucosal, rectal or lower intestinal tract. Suitable topical formulations are readily prepared for each of these areas.

For topical application to the eye, or ophthalmic use, the compounds can be formulated as micronized suspensions in isotonic, pH adjusted sterile saline, or, preferably, as solutions in isotonic, pH adjusted sterile saline, either with or without a preservative such as benzylalkonium chloride. Alternatively for the ophthalmic uses the compounds may be formulated in an ointment such as petrolatum.

For topical application to the skin, the compounds can be formulated in a suitable ointment containing the compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, the compounds can be formulated in a suitable lotion or cream containing the active compound suspended or dissolved in, for example, a mixture of one or more of the following: mineral oil, sorbitan monostearate, polysorbate 60, cetyl esters wax, cetearyl alcohol, 2-octyldodecanol, benzyl alcohol and water.

The preferred concentration of peptide construct of the present invention may be in the range of from 0.01 to 10 μg/kg in combination or mixture with an antigen or prior to exposure. However, the amount of active ingredient that may be combined with the carrier materials to produce a single dosage form will vary depending upon the host treated and the particular mode of administration. Some factors include the activity of the specific compound employed, the age, body weight, general health, sex, diet, site of administration, time of administration, rate of excretion, drug combination, and the severity of the particular disease being treated and form of administration.

Pharmaceutical methods may also be employed to control the duration of action. Controlled release preparations may be achieved through the use of polymer to complex or absorb the peptide. The controlled delivery may be exercised by selecting appropriate macromolecules (for example polyester, polyamino acids, polyvinyl, pyrrolidone, ethylenevinylacetate, methylcellulose, carboxymethylcellulose, or protamine sulfate) and the concentration of macromolecules as well as the methods of incorporation in order to control release.

For example, the inventive compounds may be incorporated into a hydrophobic polymer matrix for controlled release over a period of days. Such controlled release films are well known to the art. Particularly preferred are transdermal delivery systems. Other examples of polymers commonly employed for this purpose that may be used in the present invention include nondegradable ethylene-vinyl acetate copolymer and degradable lactic acid-glycolic acid copolymers which may be used externally or internally. Certain hydrogels such as poly(hydroxyethylmethacrylate) or poly(vinylalcohol) also may be useful, but for shorter release cycles then the other polymer releases systems, such as those mentioned above.

Alternatively, instead of incorporating these agents into polymeric particles, it is possible to entrap these materials in microcapsules prepared, for example, by coacervation techniques or by interfacial polymerization, for example, hydroxy-methylcellulose or gelatin-microcapsules and poly(methylmethacrylate) microcapsules, respectively, or in colloidal drug delivery systems, for example, liposomes, albumin microspheres, microemulsions, nanoparticles, and nanocapsules or in macroemulsions.

To be effective therapeutically for central nervous system targets, the compounds of the present invention should readily penetrate the blood-brain barrier when peripherally administered. Compounds which cannot penetrate the blood-brain barrier can be effectively administered by an intraventricular route or other appropriate delivery system suitable for administration to the brain.

The peptide constructs of the present invention may be supplied in the form of a kit, alone, or in the form of a pharmaceutical composition as described above. Administration of the immunostimulatory or adjuvant peptide and immunomodulatory compositions containing same can be conducted by conventional methods. For example, the immunogenic peptide construct can be used in a suitable diluent such as saline or water, or complete or incomplete adjuvants. The immunogen can be administered by any route appropriate for immune system stimulation, such as intravenous, intraperitoneal, intramuscular, subcutaneous, nasal, oral, rectal, vaginal, and the like. The immunogen may be administered once or at periodic intervals until, for example, a significant titer of CD4⁺ or CD8⁺ T cell and/or antibodies directed against the appropriate antigen is obtained. In particular, the antigenic peptide constructs of the invention elicit TH1 associated antibodies and other aspects of a TH1 immune response. The presence of cells may be assessed by measuring cytokine secretion specific for TH-1 (e.g., IFN-γ, IL-2) or TH-2 (e.g., IL-4, IL-10) in response to antigen-presenting cells pulsed with the immunogen. The antibody may be detected in the serum using conventional immunoassays.

As noted above, the administration of the peptide of the present invention and the immunomodulatory compositions containing same may be for either a prophylactic or therapeutic purpose. When provided prophylactically, the immunogen is provided in advance of any evidence or in advance of any symptom due to disease causing organism, tumor etc., especially in patients at significant risk for occurrence. When provided therapeutically, the immunogen is provided at (or after) the onset of the disease or at the onset of any symptom of the disease. The therapeutic administration of the immunogen serves to attenuate the disease.

Similarly, for treatment of other disease, condition or disorder, the antigenic peptide P*, will be chosen from the antigenic peptides associated with or causing the particular disease, disorder or condition, such as previously described, for example, in U.S. Pat. Nos. 5,652,342, 6,096,315, 6,093,400, 6,268,472, 6,103,239, 6,287,565, 6,111,068 and 6,258,945, other copending applications described above PCT/US98/20681, 00/41647, 00/41646 and 01/16793 or copending applications being filed simultaneously with this application or any other of the myriad known antigenic peptides associated with disease or causing disease.

According to this invention the immune response induced by this adjuvant or immunomodulatory peptide is at least predominantly directed toward at least the desired TH1 response as evidenced by the TH1 characteristic antibody IgG2a (mouse) and presumably thereby IgG3 (man). These peptide may, however, in addition to a TH1 elicited immune response, elicit a TH2 immune response, and in particular, a mixed TH1/TH2 immune response.

The present invention also contemplates vaccines. Vaccines of the present invention can be introduced into the host most conveniently by parenteral or subcutaneous injection, intramuscularly, intradermally, or orally. Any of the common liquid or solid vehicles may be employed, which are acceptable to the host and which do not have any adverse side effects on the host or any detrimental effects on the vaccine. Phosphate buffered saline (PBS), at physiological pH, e.g. pH 6.8 to 7.2, preferably pH 7, may be used as a carrier, alone or with a suitable adjuvant. The concentration of peptide construct may vary from about 0.5 to 200 μg/kg, such as about 25 μg/kg per injection, in a volume of clinical solvent generally from about 0.1 to 1 ml, such as about 0.2 ml, preclinical studies in animals, and from about 0.5 ml to about 2 ml, such as about 1 ml in humans. Multiple injections may be required after the initial injections and may be given at intervals of from about 2 to 4 weeks, for example, about 2 weeks in animals and about 4 to 8 weeks in humans, when multiple injections are given.

### EXAMPLES

### Example 1

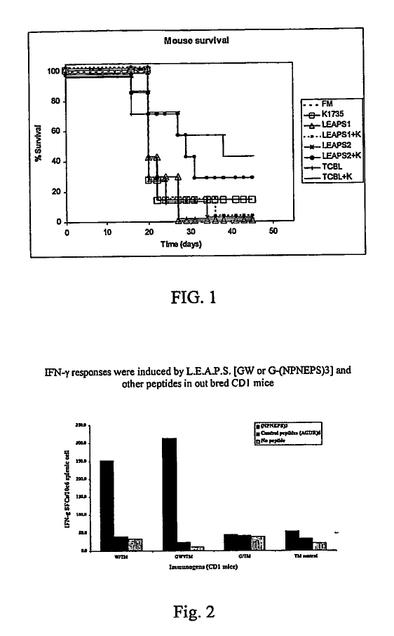

This example demonstrates the improved efficacy of a cancer vaccine utilizing peptide constructs according to the present invention as shown in FIG. 1.

Peptide constructs were prepared using as T cell binding ligand, either derG (SEQ ID NO. 7), Peptide G (SEQ ID NO. 5). The peptides are synthesized using the FMOC procedure and a double coupling protocol for the first 8 residues. Usually the peptide is prepared with the carboxyl terminus as an amide form. All of the peptides are purified using preparative HPLC, and analyzed by an analytical HPLC, amino acid analysis and mass spectrophotometer. The peptides are greater than 95%, usually greater than 98%, pure by HPLC criteria. The dry peptides are stored in vials with desiccant at −8° C.
1) Eight groups of seven mice were injected with 200 μl of the vaccine as shown in FIG. 1.
2) This was repeated after 7 days.
3) A further 7 days later, mice were challenged with 200 μl saline containing 5×10⁴ live B16 on the contralateral side to vaccination.
4) Mice were sacrificed when tumor size exceeded 15 mm in either axis.

In order more precisely model the use of clinical allogeneic vaccines, a frozen vaccine was used in the mouse model wherein K-1735 cells were thawed just prior to use. Efficacy in the protection model ranged from 40-10%. Additionally, the antigen TRP2 (a.a. 180-188), found in B16 melanoma, was engineered into L.E.A.P.S.™ constructs and was tested in various permutations in the allogeneic murine melanoma model. Optimization may be necessary.

As shown in FIG. 1, the initial experiment was repeated wherein immunization and challenge of L.E.A.P.S.™ constructs of the TRP2₁₈₀₋₁₈₈ peptide with peptide derG or J and use of a mixture of derG or J (TCBL pool) established immunostimulatory or adjuvant activity of derG. The repeated experiment produced consistent results (not shown). However, the allogeneic vaccine only showed minor efficacy. Moreover, no protection was seen when J-TRP2₁₈₀₋₁₈₈ (LEAPS 1) or the TCBL pool (derG+J) were used in isolation. Notably, J-TRP2₁₈₀₋₁₈₈ in combination with K1735 showed no protection. The results suggest that derG is the key species for both conjugates and separate entities.

In the repeated experiment, the TCBL pool was a mixture of both derG (TCBL2) and J (TCBL1). However, efficacy was not detected in the absence of allogeneic cells. On the other hand, a combination of cells resulted in a protection in a of 40% and 20% for both experiments. Since the TCBL pool was a mixture, it is difficult to assess which TCBL is active. When derG was administered in combination with cells, protection was seen at a level similar to that of TCBL (30% and 40%). Notably, derG-TRP2₁₈₀₋₁₈₈ on its own demonstrated an efficacy of 20%.

These data suggest that derG may be the active principle in the compounds tested. This peptide is present in both derG-TRP2 J-TRP2₁₈₀₋₁₈₈ (LEAPS 2) and the mixture of TCBL (J+derG), giving a similar level of protection in both cases. Interestingly, this protection is only manifest when allogeneic tumor cells are present, suggesting that derG enhances specific T-cell immunity.

Given that CTL are likely to be a major protective element in these systems, measurement of killing activity using Cr⁵¹ may be a useful bio-marker of derG activity. Clearly, allogeneic vaccination gives rise to a CTL generation capable of killing syngeneic tumor. Moreover, derG effects alone or in combination with antigen (allogeneic tumor cell vaccination) could not have occurred by chance. This is especially important when considering the differences from the parental peptide G which was much less active.

### Example 2

This example demonstrates the improved biological activity of peptide constructs according to the present invention in comparison to similar peptide constructs and conventional peptide-immunogenic carrier constructs.

Peptide constructs were prepared using as T cell binding ligand, either derG (SEQ ID NO. 7), Peptide G (SEQ ID NO. 5).

The peptides are synthesized using the FMOC procedure and a double coupling protocol for the first 8 residues. Usually the peptide is prepared with the carboxyl terminus as an amide form. All of the peptides are purified using preparative HPLC, and analyzed by an analytical HPLC, amino acid analysis and mass spectrophotometer. The peptides are greater than 95%, usually greater than 98%, pure by HPLC criteria. The dry peptides are stored in vials with desiccant at −8° C.

Evaluation of the TCBL's biological activity when used as an additive or as an immunostimulant showed desirable results in malaria protection assays. As shown in Table 4. In particular, mice were immunized twice at three week intervals with 25 μg of the subunits (W or G) used to make the L.E.A.P.S.™ construct (GW), and were challenged with 200 plasmodium yoelii sporozoites 2 weeks after the second immunization. Protective efficacy was evaluated by thin blood smears every other day starting 4 days after the challenge. Mice were considered protected if no blood stage parasites were detectable by 14 days after challenge.

TABLE 4
Table 4 The unconjugated TCBL(G), the B cell peptide
(NPNEPS)3 (W) and a mixture (G + W) were compared
to the LEAPS construct (GW) in immunized mice

| | | | |
|---|---|---|---|
| Group | Immunogen | # Protected/# tested | % protection |

Experiment 1 Inbred and outbred Mice

| | | | |
|---|---|---|---|
| 1 (A/J) | GW + TM | 7/8 | 88% |
| 2 (A/J | W + TM | 7/7 | 100% |
| 3 (A/J) | TM | 0/10 | 0% |
| 4 (Balb/C) | GW + TM | 0/8 | 0% |
| 5 (Balb/c) | W + TM | 0/8 | 0% |
| 6 (Balb/C) | TM | 0/10 | 0% |
| 7 (CD-1) | GW + TM | 7/10 | 70% |
| 8 (CD-1) | W + TM | 2/10 | 20% |
| 9 (CD-1) | TM | 3/10 | 30% |

Experiment 2 Outbred CD-1

| | | | |
|---|---|---|---|
| 1 | W + TM | 6/16 | 38% |
| 2 | G + TM | 6/16 | 38% |
| 3 | G + W + TM | 6/16 | 38% |
| 4 | GW/TM | 11/16 | 69% |
| 5 | TM | 1/16 | 7% |

The unconjugated TCBL(G) and the B cell peptide (NPNEPS)₃ (W) or a mixture of both gave less protection compared to the L.E.A.P.S.™ construct in immunized outbred CD-1 mice when administered with TiterMax™ as an adjuvant. Examination showed no detectable antibody to peptide W in the sera pools for each group (not shown).

Since additional protection was observed above adjuvant controls, a repeat experiment was done as shown in FIG. 2 to measure IFN-γ production in spleen cell cultures obtained from similarly immunized mice.

Next using another antigen with B and T cell activity two inbred strains of mice, (A/J and Balb/c) were immunized twice at three week intervals with the L.E.A.P.S.™ constructs (J-GF/SF or der-SF/GF) or with the subunits (SF/GF, J and derG) used to make heteroconjugated peptides, and were challenged with 100 Plasmodium yoelii sporozoites 2 weeks after the second immunization and protection efficacy evaluated as before. Four days before challenge, serum was collected from individual animals, pooled and analyzed immediately for antibody to SF/GF peptide sera collected from immunized and protected mice 15 days post challenge were pooled and used for IFN-γ determination by ELISA procedures.

TABLE 5
Protection studies with improved TCBL derG in inbred mice.

| | | | Antibody | Protection | IFN-γ |
|---|---|---|---|---|---|
| | | Dose | EL1SA | Protected/ | serum |
| Group | Antigen | μg | A490* | Tested (%) | pg/mL |
| A/J | | | | | |
| 1 | J-GF/SF | 5 | 1,103 | 1/10 (10%) | 0 |
| 2 | ″ | 25 | 6,121 | 10/10 (100%) | 4,000 |
| 3 | derG-GF/SF | 5 | 1,933 | 8/10 (80%) | 7,000 |
| 4 | ″ | 25 | 10,694 | 10/10 (100%) | 6,000 |
| 5 | GF/SF | 5 | 8,443 | 3/10 (30%) | 10,000 |
| 6 | ″ | 25 | 19,970 | 10/10 (100%) | 12,500 |
| 7 | J | 5 | 1 | 1/10 (10%) | nd |
| 8 | ″ | 25 | 2 | 1/10 (10%) | nd |
| 9 | der-G | 5 | 2 | 10/10 (100%) | 40,000 |
| 10 | ″ | 25 | 2 | 10/10 (100%) | 18,000 |
| 11 | TM | — | — | 0/10 (0%) | nd |
| 12 | NONE | — | — | 0/10 (0%) | nd |
| Balb/c | | | | | |
| 13 | J-GF/SF | 25 | 11,454 | 0/10 (0%) | nd |
| 14 | derG-GF/SF | 25 | 17,019 | 0/10 (0%) | nd |
| 15 | J | 25 | 2 | 0/10 (0%) | nd |
| 16 | derG | 25 | 4 | 0/10 (0%) | nd |

*ELISA 000.5 unit = serum dilution at the near OD reading is 0.5

In Table 5, J-SF/GF=DLLKNGERIEKVEGGG SFPMNEESPLGFSPEEMEAVASKFR, derG-SF/GF=DGQEEKAGVVSTGLIGGGSFPMNEESPLGFSPEEMEAVASKFR, derG=DGQEEKAGVVSTGLIGGG and SF/GF=SFPMNEESPLGFSPEEMEAVASKFR.

A substantial improvement in protection from infection was seen in the groups receiving the improved TCBL derG, whether conjugated or alone (Table 5, fifth column). Based upon the earlier results with the W and G peptide described in Table 4 and FIG. 2, IFN-γ levels in the serum pools were evaluated. However no protection was seen in the Balb/C strain (Table 5 rows 13-16). FACS analysis measuring the presence of IFN-γ⁺/CD4⁺ cells isolated from liver (the major site for infection) by perfusion and from spleen cell populations showed increased levels of IFN-γ especially for liver cells.

Another experiment similar to the first except only a single strain of mice (A/J) was used with decreasing doses of the derG in order to evaluate potency as shown in Table 6.

TABLE 6
Protection from sporozoite challenge
following peptide pretreatment.

| | | | Protection |
|---|---|---|---|
| Group A/J | Antigen | Dose μg | Protected/Tested (%) |
| 1 | GF/SF | 10 | 5/10 (50%) |
| 2 | J-GF/SF | 10 | 5/9 (56%) |
| 3 | J | 10 | 1/10 (10%) |
| 4 | GF/SF | 5 | 1/10 (10%) |
| 5 | derG-GF/SF | 5 | 1/10 (10%) |
| 6 | der-G | 5 | 10/10 (100%) |
| 7 | ″ | 2.5 | 9/10 (80%) |
| 8 | ″ | 1.25 | 6/10 (60%) |
| 9 | TM | | 1/10 (10%) |
| 10 | Naive | | 0/10 (0%) |

### Example 3

Since derG alone was able to protect A/J mice against Plasmodium yoelii sporozoites challenge, in the absence of malaria antigen, a similar experiment was conducted to evaluate the protective efficacy of derG in three other mouse strains: BALB/c (H2d), C3H/HeJ (H2K) and hybrid CAF1 (A/J×BALB/c).

Groups of 10 mice were pre-treated 2 times at 3 week intervals with 25 μg of derG in TiterMax™ adjuvant and challenged with 100 Plasmodium yoelii sporozoites. Protective efficacy was evaluated as described above.

The results indicate that derG also protects C3H/HeJ but not BALB/c and hybrid CAF1 (A/J×BALB/c).

TABLE 7
Protection from sporozoite challenge in different
mouse strains pre-treated with derG

| Group # | Mouse strains | antigen* | dose | Protection |
|---|---|---|---|---|
| 1 | A/J | derG/TM | 25 μg | 80 |
| 2 | A/J | TM | | 0 |
| 3 | A/J | Naïve | | 0 |
| 4 | BALB/c | derG/TM | 25 μg | 0 |
| 5 | BALB/c | TM | | 10 |
| 6 | BALB/c | Naïve | | 0 |
| 7 | C3H | derG/TM | 25 μg | 40 |
| 8 | C3H | TM | | 0 |
| 9 | C3H | Naïve | | 0 |
| 10 | CAF1 hybrid | derG/TM | 25 μg | 40 |
| 11 | CAF1 hybrid | TM | | 0 |
| 12 | CAF1 hybrid | Naïve | | 0 |

### Example 4

A Zosteriform Spread model of HSV infection evaluates the efficacy of antiviral drugs and vaccines wherein an improved animal model discriminates between neuroinvasive and non-neuroinvasive viruses. The scarification (abrasion)-zosteriform spread model of infection clearly demonstrates that CEL-1000 (derG) protects A/J mice from lethal HSV-1 challenge.

Groups of 4- to 6-wk old female A/J mice were subcutaneously inoculated once with 25 μg of the derG peptide emulsified in ISA51 adjuvant at a 1:1 ratio before challenge unless otherwise specified. Untreated mice and mice treated with adjuvant served as additional controls. The mice were challenged as specified in methods well known to those of ordinary skill in the art and as exemplified by Goel et al. (“The Ability of an HSV Strain to Initiate Zosteriform Spread Correlates with its Neuroinvasive Disease Potential”, Arch Virol 147:763-773 (2002)) and Goel et al., (“A Modification Of The Epidermal Scarification Model Of Herpes Simplex Virus Infection To Achieve A Reproducible And Uniform Progression Of Disease”, J Virol Meth 106; 153-8 (2002)).

In particular, hair is removed by shaving and then Nair® is applied. Twenty-fours later the depilated back of a mouse is scratched or abraded to expose the susceptible epidermal layer and virus rubbed in. To provide more detail, 10 μl of a virus suspension containing 6×10⁴ plaque forming units was pipetted onto a 0.5 cm square of skin on mouse dorsal surface, which was previously abraded using sand paper glued on a pencil eraser. In untreated animals, the primary lesion developed at the inoculation site within 3 days and spread to the associated root, followed by anterograde transport and formation of secondary lesions at sites along the dermatome of the nerves within 5 days. In severe cases, animals died within 8 days post viral challenge. Disease progression and survival were monitored for at least 10 days. Virus exhibited local site lesions, traveled to the dorsal root ganglia and then back down the neuron to cause lesions along the dermatome. The progression and the severity of the lesions was scored from 0 to 7 (death). This model allows for discrimination of the disease progression (local site, neuronal spread, extent of lesion at the dermatome, death) at which the immune system blocks viral progression. In some experiments modifications in the timing, route of administration and mouse strain were evaluated for efficacy of CEL-1000.

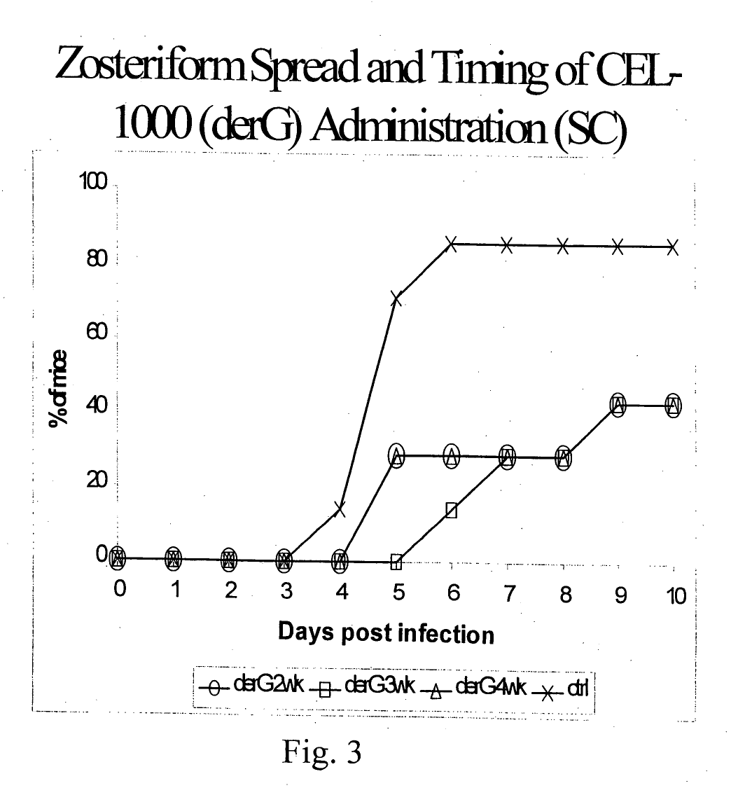

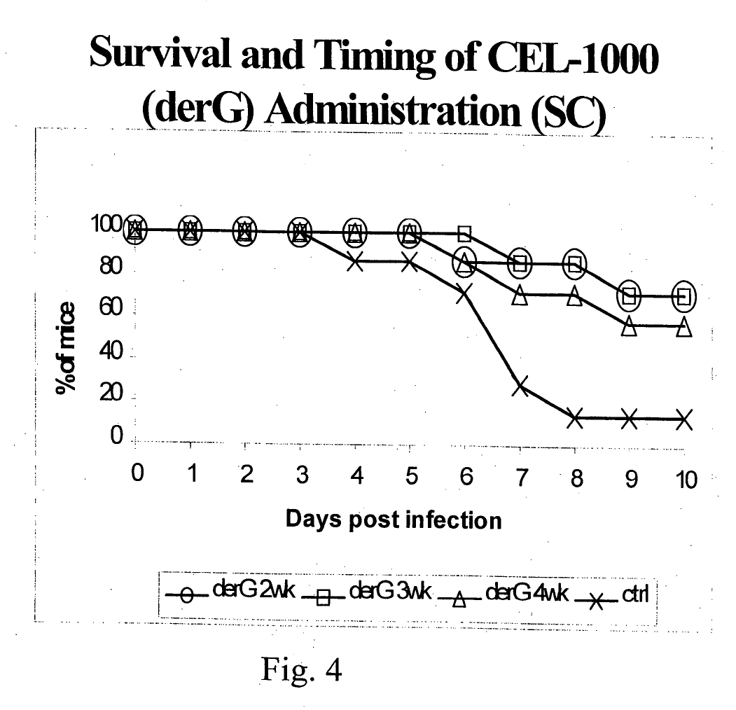

This model evaluated the timing of administration of CEL-1000 using a single dose of 25 μg. A delay in onset of symptoms reduced morbidity and mortality with zosteriform spread are shown in FIG. 3. Conversely, FIG. 4 shows that A/J mice treated with a single dose of CEL-1000 (25 μg) emulsified in Seppic ISA-51 on day 28, 14, 7 prior to challenge with HSV-1.

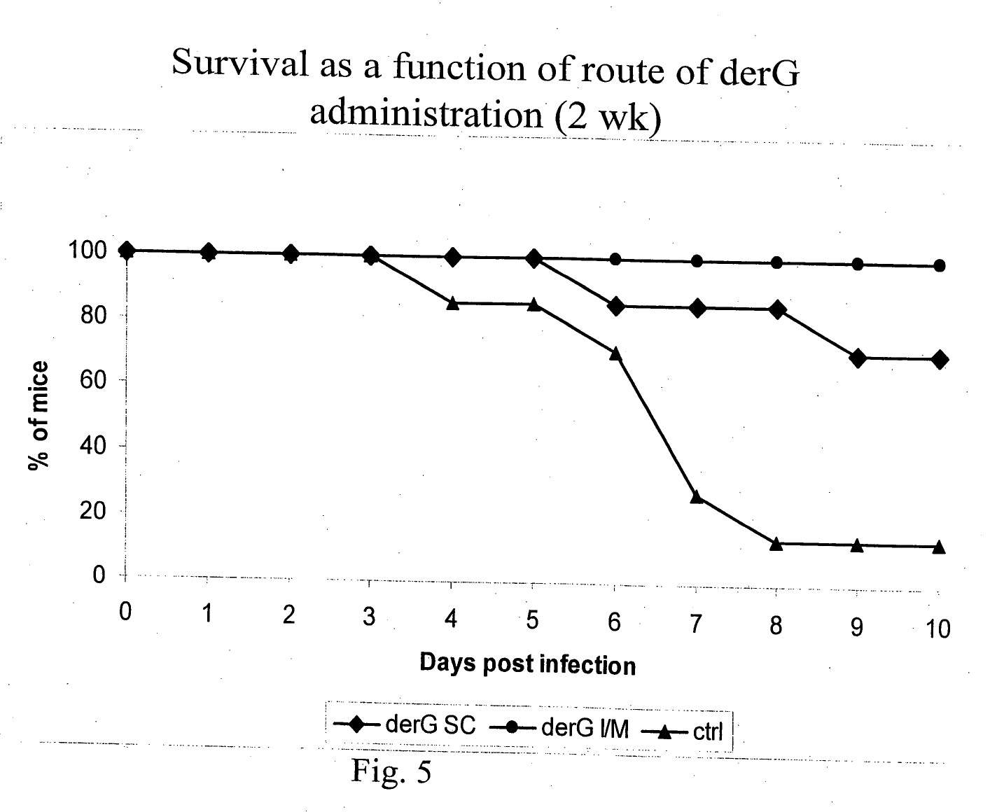

FIG. 5 shows that the same amount of CEL-1000 administered by the standard subcutaneous route emulsified with adjuvant or emulsified by a single intramuscular administration of derG in saline.

Surprisingly, intramuscular administration is more effective. Complete (100%) protection was achieved when CEL-1000 was administered intramuscularly 2 weeks prior to challenge. In addition, the dose was delivered without adjuvant, in aqueous solution, at the same concentration as the other treatments thereby suggesting that adjuvant is not necessary.

Similar protective effects of CEL-1000 were seen in other strains (both C57BL6, another inbred strain with a different MHC background and CD1 an outbred strain). Clearly, CEL-1000 (derG) prolonged survival against HSV-1 challenge.

### Prophetic Example 1

This example will demonstrate the ability of derG to accelerate and enhance the development of an immune response to a protein vaccine consisting of Botulism Neurotoxin (BoNT) recombinant heavy chain carboxyl fragment (H_{c}) antigen (representing either type A or E).

The kinetics of antibody development with or without the newly discovered immunoenhancing peptide derG is compared wherein titer will be determined for positive sera from individual mice for both class and subclass of the antigen specific antibodies. The efficacy of the anti-Type A and anti-Type E vaccines will be compared.

The new vaccine will consist of a mixture of immunogen and adjuvant.

The immunogen (BONT peptide) may be incorporated into a L.E.A.P.S.™ heteroconjugate (J-BoNT, G-BONT) or mixed with derG (derG+BONT).

The following antigens and antigenic peptides can be obtained commercially. Toxoid can be obtained from List Biological Laboratories, Campbell Calif. USA. Special pricing is available for those customers purchasing items with government funds, as List Biological Laboratories, Inc, currently has a GSA contract. This contract was issued by the Department of Veterans Affairs. The contract period is from Apr. 1, 1992 through Dec. 31, 2001, contract No. V797P-5239n and most likely will be renewed according to officials from List. In addition all the major serotypes (A-G) may be available from Dynport formerly Michigian Department of Public Health). Recombinant proteins BoNT H_{c} have been prepared by USAMRIID for most of the major serotypes A-G and contain the major portion of the nontoxic 50 kDa chain carboxyl region of the Clostridium botulism 100 kDa H chain. The intent is to use the BoNT/E H_{c} as the test material for which improvements in immunogenicity are needed and BoNT/A H_{c} as a reference material.

The formulation will include the Seppic ISA 51 adjuvant since we have previously determined that the Seppic ISA 51 was superior to the MPL with LEAPS peptide antigens.

TABLE 8
Evaluation of BoNT/E or A doses with derG

| Group # | Additives | antigen* | amount in μg |
|---|---|---|---|
| 1 | None | BoNT/E | 2.0 |
| 2 | derG* | ″ | ″ |
| 3 | None | BoNT/A** | 0.5 |
| 4 | derG | ″ | ″ |

**the E serotype is started at a five fold higher dose than the A serotype (the more potent form). Dosing and evaluations will be as in the experiment for Table 8.

Initial immunogenicity evaluations will be performed using A/J mice (Jackson Labs). Previous studies with derG performed with A/J and C57BL6 mice were shown to be encouraging. Preliminary data suggest that the Balb/c strain is not responsive to the immunoenhancing or adjuvant effect of derG and published reports suggest that the C57BL6 has a genetic alteration within their gene for IgG2a.

Groups of 10 mice will be immunized as shown in Table 8 subcutaneously in the nape of the neck on days 0, 14 and 49. Each group is composed of 5 mice (A/J). Test bleedings will be taken from the retro-orbital sinus or saphenous vein on days 0, 7, 13, 28, 42, and 63.

For the initial screening each vaccine will be evaluated at one peptide dose with or without derG. Serum is obtained during and after the immunization period. Due to potential individual mouse variation, serum from each mouse will be evaluated by ELISA for antibody reactivity towards the immunogen. The positive sera will then be evaluated for titer and antibody isotype with specific attention to IgG1 and IgG2a, indicators of Th2 and Th1 responses.

For the ELISA, reactions to the recombinant BoNT/A H_{C} and BoNT/E H_{C} antigens and the specific peptides BoNT/A₁₂₃₀₋₁₂₅₃ BoNT/E₁₂₃₀₋₁₂₅₃ will be compared to control wells coated with an unrelated control peptide(s) (Gelatin hydrolysate avg 3500 kDa) or protein (BSA) at 1 μg/mL. The control wells will allow correction for any non-specific signal due to binding to uncoated wells or to unrelated peptide antigen. This effect can be substantial with earlier immune responses when large amounts of heteroclictic antibodies are generated.

The antibody positive sera produced in response to the various peptide immunogens will be analyzed for the titer to determine the actual efficacy of the immunization as well the specific isotype responses for IgG1 and IgG2 using only optimal antigen. A control for non-specific binding; and cross reactivity of the antisera, by analysis of BoNT/E immunized samples on BoNT/A proteins and vice versa will also be analyzed. IgG1 and IgG2 isotype responses will be used as indicators for the Th1/Th2 bias of the immune response.

Subsequent experiments will compare the effect of derG on the response to smaller doses of vaccine immunogen (0.5, 0.1, 0.02 mg).

The invention being thus described, it will be obvious that the same may be varied in many ways. Such variations are not to be regarded as a departure from the spirit scope of the invention and all such modifications are intended to be included within the scope of the following claims.

We claim:

1. A method for treating cancer, autoimmune, or transplant conditions, comprising administering a polypeptide as shown in SEQ ID NO.'s 1-28 to an animal in need thereof.

2. The method of claim 1, wherein the polypeptides are used as adjuvants or immunomodulatory agents for tumors.

3. The method of claim 1, wherein the polypeptides are used as adjuvants or immunomodulatory agents for self antigens.

4. The method of claim 1, wherein the polypeptides are administered as a mixture with or separate from the antigen given at the same time.

5. A method for determining an immunomodulatory agent as a prophylactic or a therapeutic agent with applications in cancers, autoimmune and transplant rejection conditions, comprising the steps of evaluating SEQ ID NO.'s 1-28 using Microarray technology.

6. A method for treating infectious conditions or allergies caused by foreign (i.e. not self) eukaryotic organisms, comprising administering a polypeptide as shown in SEQ ID NO.'s 1-28 to an animal in need thereof.

7. The method of claim 6, wherein the polypeptides are used as adjuvants or immunomodulatory agents for infectious conditions or allergies caused by foreign eukaryotic organisms.

8. The method of claim 6, wherein the polypeptides are used as adjuvants or immunomodulatory agents for infectious conditions or allergies caused by foreign eukaryotic organisms without antigen.

9. The method of claim 6, wherein the polypeptides are administered as a mixture with or separate from the antigen given at the same time.

10. The method of claim 6, wherein the polypeptides are formulated into compositions suitable for military applications.

11. A method for determining an immunomodulatory agent as a prophylactic or a therapeutic agent for treating infectious conditions or allergies caused by foreign (i.e. not self) eukaryotic organisms, comprising the steps of evaluating SEQ ID NO.'s 1-28 using Microarray technology.

12. A method for treating infectious conditions or allergies caused by parasitic organisms, comprising administering a polypeptide as shown in SEQ ID NO.'s 1-28 to an animal in need thereof.

13. A method for treating infectious conditions caused by prokaryotic organisms or non-living agents such as viruses, phages and prions, comprising administering a polypeptide as shown in SEQ ID NO.'s 1-28 to an animal in need thereof.

14. The method of claim 13, wherein the polypeptides are used as adjuvants or immunomodulatory agents for infectious conditions or allergies caused by prokaryotic organisms or non-living agents such as viruses, phages and prions.

15. The method of claim 13, wherein the polypeptides are used as adjuvants or immunomodulatory agents for infectious conditions or allergies caused by prokaryotic organisms or non-living agents such as viruses, phages and prions without antigen.

16. The method of claim 13, wherein the polypeptides are administered as a mixture with or separate from the antigen given at the same time.

17. The method of claim 13, wherein the polypeptides are formulated into compositions suitable for military applications.

18. A method for determining an immunomodulatory agent as a prophylactic or a therapeutic agent with applications in treating disease or infectious conditions caused by prokaryotic organisms or non-living agents such as viruses, phages and prions, comprising the steps of evaluating SEQ ID NO.'s 1-28 using Microarray technology.
